(19) United States
(12) Patent Application Publication
(10) Pub. No.: US 2023/0242606 A1
(43) Pub. Date: Aug. 3, 2023

(54) CXCL9 AND VARIANTS THEREOF FOR IMMUNOTHERAPY OF CANCER DISEASES

(71) Applicant: TECHNION RESEARCH & DEVELOPMENT FOUNDATION LIMITED, Haifa (IL)

(72) Inventors: Nathan KARIN, Haifa (IL); Ghada JARROUS, Haifa (IL)

(21) Appl. No.: 18/010,105

(22) PCT Filed: Jun. 21, 2021
(86) PCT No.: PCT/IL2021/050751

§ 371 Date: Dec. 13, 2022

Related U.S. Application Data
(60) Provisional application No. 63/150,629, filed on Feb. 18, 2021.
(60) Provisional application No. 63/041,940, filed on Jun. 21, 2020.

(51) Int. Cl. C07K 14/52; A61P 35/00

(52) U.S. Cl. CPC C07K 14/521; A61P 35/00; C07K 2319/30; A61K 38/00

(57) ABSTRACT

The invention provides a modified CXCL9 polypeptide comprising an insertion of an additional amino acid at the N-terminus of a corresponding wild type CXCL9, a pharmaceutical composition comprising the same and a method for the production thereof and of using the same for treating cancer. Further provided nucleic acids encoding the modified CXCL9 polypeptides of the invention, vectors and host cells comprising the same.

### BACKGROUND OF THE INVENTION

While many chemokines produced by cancer cells that also possess their receptors, clearly support tumor growth and suppress anti-cancer immune reactivity, the CXCR3 ligand CXCL10, is thought to attenuate tumor growth by eliciting an anti-cancer immune response. CXCL10 may also directly inhibit tumor growth. CXCR3 is a chemokine receptor with three ligands: CXCL9, CXCL10 and CXCL11. The different CXCR3 ligands may differ in their biological functions.

Several studies showed that CXCL9 and CXCL10, particularly CXCL10 produced by tumor or host cells can recruit CXCR3+ tumor-infiltrating CD4+ T cells, CD8+ T cells and NK cells that are associated with tumor suppression. It was shown that anti PD-1 efficacy is reduced in CXCR3KO mice, and suggested that the interaction between CXCL9, largely produced by CD103+ dendritic cells (DC) at the tumor site, and CXCR3 on CD8+ T cells enhances anti PD-1 efficacy (Chow et al, Immunity 2019, 50 1498-1512 e5).

The role of CXCL9 in cancer therapy was not studied. The in vivo stability of chemokines, particularly CXCL9 is very limited. It was shown that generation of stabilized chemokines as fusion proteins with Ig FC leads to prolong in vivo half-life (Barsheshet et al PNAS 2017).

The in vivo activity of CXCL10 and CXCL9, particularity at tumor sites, is regulated by Dipeptidyl Peptidase 4 (DPP4, also known as CD26) that acts on the proline at position 2 and induces cleavage of the two N-terminus amino acids, resulting in a non-functional CXCL9, or CXCL10 that may also act as an antagonist chemokine to CXCL9 and CXCL10, respectively, as the truncated protein may inhibit intact chemokines.

Systemic targeting of DPP4 may thus be beneficial in inhibiting cancer development, but may hold major side effects due to the critical role of this enzyme in regulating different biological functions, among them glucose metabolism.

There is a need to develop modified CXCL9 polypeptides that will be stable and efficient as an anticancer drug. Further, there is a need to develop a stabilized and modified CXCL9 that will be resistant to DPP4 cleavage.

### SUMMARY OF THE INVENTION

According to some embodiments, there is provided an advantageous modified CXCL9 polypeptide, which includes one or more-point mutations and/or insertion compared to a wild-type (non-modified) CXCL9. According to some embodiments, the novel, non-naturally occurring, modified CXCL9 disclosed herein is advantageous, as it is stable, easy to produce, and exhibit a desired biological activity, as further detailed herein. Further provided are nucleic acids encoding for the modified CXCL9 polypeptide, methods for the preparation of the modified CXCL9, compositions comprising the same and uses thereof in treating various medical conditions, in particular, cancer.

In some embodiments, there is provided a modified CXCL9 polypeptide, comprising an insertion of one or more additional amino acids at the N-terminus of a corresponding wild type CXCL9 as denoted by SEQ ID NO: 1.

In some embodiments, there is provided a modified CXCL9 polypeptide, comprising an insertion of an additional amino acid at the N-terminus of a corresponding wild type CXCL9. In some embodiments, the additional amino acid is any amino acid. In some embodiments, the additional amino acid is glutamine, asparagine, pyroglutamate, glutamic acid or proline. In some embodiments, the wild type CXCL9 is of human origin. In some embodiments, the modified CXCL9 polypeptide comprises an amino acid sequence as denoted by any one of SEQ ID NOs: 2-4.

In some embodiments, the modified CXCL9 polypeptide described herein is linked to an immunoglobulin (Ig) molecule or a fragment of an Ig molecule. The immunoglobulin is in some embodiments, IgG-Fc: hinge-ch2-ch3 denoted by SEQ ID No: 5. In some embodiments, the modified CXCL9 polypeptide described herein which is linked to an immunoglobulin (Ig) molecule or a fragment of an Ig molecule further comprises a linker between the modified CXCL9 and the immunoglobulin molecule or the fragment thereof. In some embodiments, the immunoglobulin or the fragment thereof is of human origin. In some embodiments, the linker comprises a stretch of one or more Glycine amino acids (poly G) or a stretch of Glycine and Serine amino acids (poly GS). In some embodiments, the poly GS is GGGGSGGGGSGGGGS (SEQ ID No: 6).

In some embodiments, the modified CXCL9 polypeptide is capable of binding to CXCR3 receptor. In some embodiments, the modified CXCL9 polypeptide is capable of inducing CD8+ T cells.

In some embodiments, there is provided a fusion protein comprising CXCL9 polypeptide (that may be wild type or modified) conjugated to an immunoglobulin molecule or a fragment of an Ig molecule. In some embodiments, the immunoglobulin or the fragment thereof is IgG-Fc: hinge-ch2-ch3. In some embodiments, the CXCL9, the immunoglobulin molecule or the fragment thereof are of human origin. In some embodiments, the fusion protein further comprises a linker between the CXCL9 and the immunoglobulin or the fragment thereof. The linker may be a stretch of one or more Glycine amino acids (poly G) or a stretch of Glycine and Serine amino acids (poly GS). In some embodiments, the poly GS is GGGGSGGGGSGGGGS (SEQ ID No: 6).

In some embodiments, the fusion protein is capable of binding to CXCR3 receptor. In some embodiments, the fusion protein is capable of inducing CD8+ T cells. CXCL9 may induce (potentiate) the activity of CD8+ T cells by eliciting the levels of interferon gamma (IFN-g), tumor necrosis factor alpha (TNFa), Granzyme-B, perforin, and Interleukin 2 (IL-2)

In some embodiments, there is provided a method of treating cancer in a subject in need thereof, the method comprising administering to the subject in need thereof a therapeutically amount of the modified CXCL9 polypeptide or the fusion protein of the invention or of a pharmaceutical composition comprising the same. In some embodiments, there is provided a pharmaceutical composition comprising the modified CXCL9 polypeptide or the fusion protein of the invention and a pharmaceutically acceptable carrier. In some embodiments, the pharmaceutical composition is suitable for use in treating cancer.

In some embodiments, there is provided a nucleic acid molecule encoding the modified CXCL9 polypeptide or the fusion protein of the invention. In some embodiments, there is provided vector comprising the nucleic acid molecule described herein. In some embodiments, the vector is an expression vector, further comprising one or more regulatory sequences.

In some embodiments, the vector or the nucleic acid may be used in treating cancer in a subject in need thereof. In some embodiments, the vector or the nucleic acid may be used in treating cancer in a subject in need thereof. In some embodiments, there is provided a method of treating cancer in a subject in need thereof, the method comprising administering to the subject in need thereof a therapeutically amount of the nucleic acid molecule or of the vector of the invention. In some embodiments, there is provided a host cell comprising the nucleic acid molecule of the invention. In some embodiments, there is provided a host cell transformed or transfected with the vector of the invention. In some embodiments, there is provided a host cell comprising the modified CXCL9 polypeptide or the fusion protein of the invention. In some embodiments, there is provided a method of producing the modified CXCL9 polypeptide, the method comprising: (i) culturing the host cells comprising the nucleic acid molecule of the invention under conditions such that the polypeptide comprising the modified CXCL9 is expressed; and (ii) recovering the modified CXCL9 polypeptide from the host cells or from the culture medium.

In some embodiments of the invention, the modified CXCL9 polypeptide described above is linked to an immunoglobulin or to a fragment thereof. In some embodiments of the invention, there is provided a WT CXCL9 polypeptide linked to an immunoglobulin or to a fragment thereof.

In some embodiments of the invention, there is provided a stabilized CXCL9 chemokine which is a CXCL9-Ig fusion polypeptide that optionally includes a poly GS linker.

In some embodiments of the invention, there is provided a modified CXCL9 polypeptide comprising an insertion of one or more tandem repeats of the peptide “GGGGS” SEQ ID No: 7 (four glycines and one serine) at the C-terminus of a corresponding WT CXCL9 polypeptide. The insertion of the one or more GGGGS units is referred in here to as polyGS.

In an embodiment of the invention, there is provided a modified CXCL9 polypeptide comprising an insertion of a stretch of one or more units of Glycine and Serine amino acids (poly GS) at the C-terminus of a corresponding WT CXCL9 polypeptide.

In some embodiments of the invention, the modified CXCL9 polypeptide described above is linked to an immunoglobulin or to a fragment thereof. In some embodiments of the invention, there is provided a WT CXCL9 polypeptide linked to an immunoglobulin or to a fragment thereof. In some embodiments of the invention, there is provided a WT CXCL9 polypeptide linked to a non-proteinaceous moiety. In some embodiments of the invention, there is provided a modified CXCL9 polypeptide linked to a non-proteinaceous moiety.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 shows that CXCL9 inhibits proliferation Hela (human cervical cells cancer). Hela cells were seeded in 96 well plate in RPMI medium supplemented with 10% FCS, penicillin, streptomycin and glutamic acid (2×10⁴ cells per well). 24 h after seeding, the medium was replaced with fresh one supplemented with different concentration of mouse CXCL9 (Peprotech Cat #250-18) as indicated in the graph, or without treatment (WO). 24 hours later XTT assay was performed according to the manufactory instructions (Biological Industries, cat #20-300-1000). The OD measurements were taken after two hours incubation with the XTT substrate. The assay was performed with six well for each treatment.

FIG. 2A-2C show tumor progression and mortality analysis in C57Bl/6 mice treated with CXCL9-Ig versus the control group Mice (14 females at age of 8 weeks) were injected subcutaneously with 3.5×10⁵ Ret cells at the back. On day 3, mice were separated into 5 groups of 7 females each. Each group was treated (3 time a week, 40 μg/mouse) with either CXCL9-Ig, or with isotype matched control IgG (calibrated according molar adjustment). On day 9, a single mouse with no tumor development has been subtracted from each group. On day 23, therapy was terminated and mice were continued to be followed for mortality. FIG. 2A shows tumor size as mean size±SD (length×width×height)×0.52. FIG. 2B shows scattered analyses on day 17. FIG. 2C shows mortality curve. *P≤0.05 was considered as significant.

### DESCRIPTION OF THE DETAILED EMBODIMENTS

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As referred to herein, the terms “polynucleotide molecules”, “oligonucleotide”, “polynucleotide”, “nucleic acid” and “nucleotide” sequences may interchangeably be used. The terms are directed to polymers of deoxyribonucleotides (DNA), ribonucleotides (RNA), and modified forms thereof in the form of a separate fragment or as a component of a larger construct, linear or branched, single stranded (ss), double stranded (ds), triple stranded (ts), or hybrids thereof. The polynucleotides may be, for example, or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions. As used herein, nucleotides (A, G, C or T) and nucleotide sequences are marked in lowercase letters (a, g, c or t).

The terms “polypeptide,” “peptide” and “protein” are used interchangeably herein to refer to a polymer of amino acid residues. The terms also apply to amino acid polymers in which one or more amino acid residue is an artificial chemical analogue of a corresponding naturally occurring amino acid, as well as to naturally occurring amino acid polymers. In some embodiments, one or more of amino acid residue in the polypeptide, can contain modification, such as but be not limited only to, glycosylation, phosphorylation or disulfide bond shape. Also provided are conservative amino acid variants of the peptides and protein molecules disclosed herein. Variants according to the invention also may be made that conserve the overall molecular structure of the encoded proteins or peptides. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. As used herein, Amino acids and peptide sequences are marked using conventional Amino Acid nomenclature (single letter or 3-letters code). For example, amino acid “Serine” may be marked as “Ser” or “S” and amino acid “Cysteine” may be marked as “Cys” or “C”.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A or a) forms a base pair with thymine (T or t) and guanine (G or g) with cytosine (C or c). In RNA, thymine is replaced by uracil (U or u). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors, plasmids but should not be seen as being limited thereto. The term “regulatory sequence” in some embodiments, refers to DNA sequences, which are necessary to effect the expression of coding sequences to which they are operably linked (connected/ligated). The nature of the regulatory sequences differs depending on the host cells. For example, in prokaryotes, regulatory/control sequences may include promoter, ribosomal binding site, and/or terminators. For example, in eukaryotes regulatory/control sequences may include promoters, terminators enhancers, transactivators and/or transcription factors. A regulatory sequence which is “operably linked” to a coding sequence is ligated in such a way that expression of the coding sequence is achieved under suitable conditions. In some embodiments, a “Construct” or a “DNA construct” refer to an artificially assembled or isolated nucleic acid molecule which comprises a coding region of interest and optionally additional regulatory or non-coding sequences.

As used herein, the term “vector” refers to any recombinant polynucleotide construct (such as a DNA construct) that may be used for the purpose of transformation, i.e. the introduction of heterologous DNA into a host cell. One exemplary type of vector is a “plasmid” which refers to a circular double stranded DNA loop into which additional DNA segments can be ligated. Another exemplary type of vector is a viral vector, wherein additional DNA segments can be ligated into the viral genome. Certain vectors are capable of autonomous replication in a host cell into which they are introduced. The term “Expression vector” refers to vectors that have the ability to incorporate and express heterologous nucleic acid fragments (such as DNA) in a foreign cell. In other words, an expression vector comprises nucleic acid sequences/fragments (such as DNA, mRNA), capable of being transcribed or expressed in a target cell. Many viral, prokaryotic and eukaryotic expression vectors are known and/or commercially available. Selection of appropriate expression vectors is within the knowledge of those having skill in the art. The expression vectors can include one or more regulatory sequences.

As used herein, a “primer” defines an oligonucleotide which is capable of annealing to (hybridizing with) a target nucleotide sequence, thereby creating a double stranded region which can serve as an initiation point for DNA synthesis under suitable conditions.

As used herein, the term “transformation” refers to the introduction of foreign DNA into cells. The terms “transformants” or “transformed cells” include the primary transformed cell and cultures derived from that cell regardless to the number of transfers. All progeny may not be precisely identical in DNA content, due to deliberate or inadvertent mutations. Mutant progeny that has the same functionality as screened for in the originally transformed cell are included in the definition of transformants.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, injection, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

The terms “upstream” and “downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

As used herein, the term “treating” includes, but is not limited to one or more of the following: abrogating, ameliorating, inhibiting, attenuating, blocking, suppressing, reducing, delaying, halting, alleviating or preventing symptoms associated with a condition. Each possibility represents a separate embodiment of the present invention. In some embodiments, the condition is a cancer. In some exemplary embodiments, the condition may be selected from, melanoma or metastatic melanoma and the like.

The term “CXCL9” is interchangeable with any alternative name or synonym of this protein known in the art. The term refers to a protein or polypeptide, primarily to a human protein. The terms further refer to a nucleic acid encoding for the corresponding polypeptide. The amino acid sequences and encoding nucleotide sequences of CXCL9 are well known in the art. Nucleic acid sequences can be retrieved in public databases like NCBI. In some embodiments, the Homo sapiens Wild Type (WT) CXCL9 corresponds to SEQ ID NO: 8.

The term “wild type CXCL9”, “WT CXCL9”, “naturally occurring CXCL9” and “un-modified CXCL9” may interchangeably be used. The terms refer to the naturally occurring form of CXCL9 (i.e., an endogenous, non-mutated CXCL9 or full-length CXCL9). In some embodiments, the WT-CXCL9 is of a mammalian origin. In some embodiments, the WT-CXCL9 is of human origin. In some embodiments, the WT-CXCL9 of human origin has an amino acid sequence as denoted by SEQ ID NO:8. The polynucleotide sequence as set forth in SEQ ID NO: 9 corresponds to the cDNA encoding human WT CXCL9 as set forth in SEQ ID NO: 8.

As used herein the terms “modified CXCL9”, “mutated CXCL9”, “non-naturally occurring CXCL9”, may interchangeably be used. The terms relate to a mutated/modified form of the corresponding wild-type (WT) or natural form of the CXCL9. In some embodiments, the CXCL9 is of human origin and it is termed “modified hCXCL9”, “mutated hCXCL9”, “non-naturally occurring hCXCL9” or “modified CXCL9”, “mutated CXCL9”, “non-naturally occurring CXCL9”. In some embodiments, the CXCL9 is of mammalian origin. In some embodiments, the modified CXCL9 differs from the corresponding wild type CXCL9 by at least one mutation selected from amino acid substitution(s), insertion(s) and/or deletions(s). In some embodiments of the invention, the modified CXCL9 polypeptide may be conjugated to an immunoglobulin (Ig) or a fragment thereof. In some embodiments of the invention, the Ig, which may be an IgG or the fragment thereof is without limitation, IgG-Fc: hinge-ch2-ch3. Such a conjugated modified CXCL9 polypeptide is also defined here as modified CXCL9 polypeptide and may be interchangeably defined as modified CXCL9-Ig polypeptide. In some embodiments of the invention, the term “modified CXCL9-Ig polypeptide” or “modified CXCL9 polypeptide”, refers also to CXCL9 polypeptide or CXCL9-Ig polypeptide with a poly G or poly GS linker. In some embodiments, the terms “modified CXCL9-Ig polypeptide” or “modified CXCL9 polypeptide” also include a chimera or a conjugate of WT CXCL9 polypeptide or mutant CXCL9 polypeptide conjugated to an Ig, or to non-proteinaceous moieties (e.g., PEG), with or without a linker. In some embodiments, the modified CXCL9 is a human WT CXCL9 with an additional amino acid inserted at the N-terminus of CXCL9. The inserted amino acid may be in some embodiments, any amino acid. In some embodiments, the inserted amino acid is glutamine, asparagine, pyroglutamate, glutamic acid or proline. In some embodiments, the additional amino acid is Phenylalanine, Leucine, Isoleucine, Valine, Tyrosine, Histidine, Lysine, aspartate, glutamate, Arginine or Glycine.

The examples of the invention show that chemokine CXCL9 could be used to treat or suppress cancer diseases. In some embodiments of the invention, CXCL9, CXCL9-Ig and the modified CXCL9 polypeptide induce anti-tumor CD8+ T cells, and by so doing suppress cancer diseases, for example without limitation, cervical cancer, melanoma and colorectal cancer. In some embodiments of the invention, CXCL9, CXCL9-Ig and modified CXCL9 polypeptide limit or prevent cancer. In some embodiments of the invention, CXCL9, CXCL9-Ig and modified CXCL9 polypeptide limit, suppress or prevent cancer diseases, such as colorectal cancer, ovarian carcinoma, osteosarcoma (OS), cervical cancer, melanoma, lung cancer, head and neck cancer and hepatocellular carcinoma (HCC).

In some embodiments of the invention, there is provided a CXCL9-Ig based fusion protein. In some embodiments of the invention, there is provided a CXCL9-Ig based fusion protein, wherein the CXCL9 is a modified human CXCL9 polypeptide.

In some embodiments, the modified CXCL9 polypeptide or the modified CXCL9-Ig polypeptide the invention is capable of binding to CXCR3 receptor.

In some embodiments, the modified CXCL9 polypeptide or the modified CXCL9-Ig polypeptide is capable of inducing CD8+ T cells. By “inducing” it is meant the potentiation or amplification of the activity of the cells including but not limited to cytotoxicity.

In some embodiments, the sequence of a human CXCL9 wild type (WT) is as set forth below at SEQ ID No. 8:

hCXCL9-WT
Protein sequence
(SEQ ID No: 8)
MKKSGVLFLLGIILLVLIGVQGTPVVRKGRCSCISTNQGT
IHLQSLKDLKQFAPSPSCEKIEIIATLKNGVQTCLNPDSAD
VKELIKKWEKQVSQKKKQKNGKKHQKKKVLKVRKSQRSRQKKTT.

In some embodiments of the invention, the cDNA sequence encoding the hCXCL9 WT is as set forth in SEQ ID No: 9:

(SEQ ID No: 9)
atgaagaaaa gtggtgttct tttcctcttg ggcatcatct tgctggttct gattggagtg
caaggaaccc cagtagtgag aaagggtcgc tgttcctgca tcagcaccaa ccaagggact
atccacctac aatccttgaa agaccttaaa caatttgccc caagcccttc ctgcgagaaa
attgaaatca ttgctacact gaagaatgga gttcaaacat gtctaaaccc agattcagca
gatgtgaagg aactgattaa aaagtgggag aaacaggtca gccaaaagaa aaagcaaaag
aatgggaaaa aacatcaaaa aaagaaagtt ctgaaagttc gaaaatctca acgttctcgt
caaaagaaga ctaca

In some embodiments of the invention, there is provided a modified hCXCL9 polypeptide that includes a poly G or a poly GS chain. In some embodiments, there is provided a modified CXCL9 polypeptide comprising an insertion of a stretch of Glycine and Serine amino acids, which may be a unit chain of 4 glycines and one serine (poly GS) at the C-terminus of a corresponding WT CXCL9 polypeptide.

As used herein, a “stretch” of “amino acids” means a plurality of amino acids arranged in a chain, each of which is joined to a preceding amino acid by a peptide bond. The amino acids of the chain may be naturally or non-naturally occurring, or may comprise a mixture thereof. In some embodiments, each “stretch”, contains two or more amino acid residues that are adjacent to each other or close to each other (i.e., in the primary or tertiary structure of the amino acid sequence).

In some embodiments of the invention, the modified hCXCL9 polypeptide with a poly G comprises a sequence as set forth in SEQ ID No: 10:

Mutant 1—hCXCL9-polyGS—Insertion Poly GS Sequence at the hCXCL9 C-Terminus.

Protein sequence of the hCXCL9-polyGS mutant

(SEQ ID No: 10)
MKKSGVLFLLGIILLVLIGVQGTPVVRKGRCSCISTNQGT
IHLQSLKDLKQFAPSPSCEKIEIIATLKNGVQTCLNPDSAD
VKELIKKWEKQVSQKKKQKNGKKHQKKKVLKVRKSQRSRQKKTTGGGGS
GGGGSGGGGS

In some embodiments of the invention, the cDNA sequence encoding the modified CXCL9 polypeptide as set forth in SEQ ID No: 10 is set forth in SEQ ID No: 11.

cDNA Sequence of the hCXCL9-polyGS mutant:
(SEQ ID No: 11)
atgaagaaaa gtggtgttct tttcctcttg ggcatcatct tgctggttct gattggagtg
caaggaaccc cagtagtgag aaagggtcgc tgttcctgca tcagcaccaa ccaagggact
atccacctac aatccttgaa agaccttaaa caatttgccc caagcccttc ctgcgagaaa
attgaaatca ttgctacact gaagaatgga gttcaaacat gtctaaaccc agattcagca
gatgtgaagg aactgattaa aaagtgggag aaacaggtca gccaaaagaa aaagcaaaag
aatgggaaaa aacatcaaaa aaagaaagtt ctgaaagttc gaaaatctca acgttctcgt
caaaagaagactacaGGCGGAGGTGGCTCTGGCGGTGGCGGATC
GGGCGGAGGTGGCTCT

In some embodiments, the CXCL9 or the modified CXCL9 polypeptide includes an IgG-Fc: hinge-ch2-ch3.

In some embodiment, the IgG-Fc: hinge-ch2-ch3 is a human IgG-Fc: hinge-ch2-ch3 as set forth in SEQ ID No: 5.

hIgG-Fc: hinge-ch2-ch3
Protein:
SEQ ID No: 5
EPKSCDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQVSLTCLVK
GFYPS
DIAVEWESNGQPENNYKTTPPVLDSDGSFFLYSKLTVDKSRWQQGNVFSCSVMHEA
LHNHYTQKSLSLSPGK
cDNA of the hIgG-Fc: hinge-ch2-ch3
(SEQ ID No: 24)
gagcccaaatcttgtgacaaaactcacacatgcccaccgtgcccagcacctgaactcctg
gggggaccgtcagtcttcctcttccccccaaaacccaaggacaccctcatgatctcccgg
acccctgaggtcacatgcgtggtggtggacgtgagccacgaagaccctgaggtcaagttc
aactggtacgtggacggcgtggaggtgcataatgccaagacaaagccgcgggaggagcag
tacaacagcacgtaccgtgtggtcagcgtcctcaccgtcctgcaccaggactggctgaat
ggcaaggagtacaagtgcaaggtctccaacaaagccctcccagcccccatcgagaaaacc
atctccaaagccaaagggcagccccgagaaccacaggtgtacaccctgcccccatcccgg
gaggagatgaccaagaaccaggtcagcctgacctgcctggtcaaaggcttctatcccagc
gacatcgccgtggagtgggagagcaatgggcagccggagaacaactacaagaccacgcct
cccgtgctggactccgacggctccttcttcctctatagcaagctcaccgtggacaagagc
aggtggcagcaggggaacgtcttctcatgctccgtgatgcatgaggctctgcacaaccac
tacacgcagaagagcctctccctgtccccgggtaaa

In some embodiments of the invention, there is provided a modified hCXCL9-with poly GS and IgG-Fc. In some embodiments, the Ig is IgG-Fc: hinge-CH2-CH3. In some embodiments, IgG-Fc: hinge-CH1-CH2-CH3 may be used.

In some embodiments, the human CXCL9 or the modified CXCL9 polypeptide includes a human IgG-Fc sequence or fragment thereof resulting in CXCL9-Ig based fusion protein or modified CXCL9-Ig polypeptide.

In some embodiments of the invention, a polyG or polyGS linker may be added to the CXCL9 or the mutated CXCL9. In some embodiments, a sequence of one or more repeated GGGGS (SEQ ID No: 7) is added. The linkers will be inserted between the Fc and the c-terminal part of CXCL9. The linkers may be a GGGGS unit as set forth in SEQ ID No: 7, two or more repeated units, such as for example, three units as set forth in GGGGSGGGGSGGGGS (SEQ ID No: 6) as described in Shen, Z et al Anal Chem 77, 6834-6842 (2005), and Kim et al PloS one 9, e113442 (2014). In some embodiments, a linker comprises of at least two glycine is added. In some embodiments, a linker comprises between 2-20 glycine is added.

In some embodiments, the invention relates to any chemokine having proline at position 2, such as, hCXCL9 or hCXCL10. In some embodiments, there is provided a method of blocking DPP4 proteolytic cleavage at the N-terminus of hCXCL9 or any other chemokine having a proline at position 2 (P2), such as hCXCL10, by insertion of one amino acid at the N-terminus to move the proline at position 2 to position 3 (DPP4 is an exopeptidase that cleaves specifically proline at position 2 at the C-terminus). In some embodiments, the inserted amino acid is glutamine, asparagine, pyroglutamate, glutamic acid or proline. Potentially, all of the amino acids that can be inserted to the N-terminus without interfering the signal peptidase cleavage (to remove the N-terminus) can be used.

In some embodiments, there is provided a human modified CXCL9 polypeptide, in which an amino acid, which can be any amino acid, is inserted before the proline at position 2.

In some embodiments, the inserted amino acid is glutamine, asparagine, pyroglutamate, glutamic acid or proline. The insertion causes the proline to move to position 3 thereby preventing the cleavage by DPP4. In some embodiments, glutamine is inserted at the N-terminus of the hCXCL9. According to some embodiments of the invention, the sequence of such a modified CXCL9 polypeptide is as shown in SEQ ID No: 1, SEQ ID No: 2, SEQ ID No: 3, or SEQ ID No: 4:

Mutant-1: hCXCL9 with an Insertion of X Amino Acid (X May be any Amino Acid) at N-ter

Protein sequence including the signal peptide:
(SEQ ID No. 1)
MKKSGVLFLLGIILLVLIGVQGXTPVVRKGRCSCISTNQGT
IHLQSLKDLKQFAPSPSCEKIEIIATLKNGVQTCLNPDSADVKELIKKW
EKQVSQKKKQKNGKKHQKKKVLKVRKSQRSRQKKTT.

The signal peptide of the CXCL9 is in italics. Xaa inserted immediately after the signal peptide cleavage site that considers as position 0.

In some embodiments, the polypeptide comprises the sequence of SEQ ID No. 2:

Mutant 2: hGln-CXCL9—Insertion Gln at Position 0 of the hCXCL9 N-Terminus

Protein sequence of the Gln-CXCL9-Ig mutant
(SEQ ID No. 2)
MKKSGVLFLLGIILLVLIGVQGQTPVVRKGRCSCISTNQGT
IHLQSLKDLKQFAPSPSCEKIEIIATLKNGVQTCLNPDSAD
VKELIKKWEKQVSQKKKQKNGKKHQKKKVLKVRKSQRSRQKKTT.

In some embodiments, and as an example, the sequence of the cDNA encoding hCXCL19 with an—insertion of GLN at N-terminus is as set forth below at SEQ ID No. 12:

cDNA sequence of the hGln-CXCL9 mutant:
(SEQ ID No. 12)
atgaagaaaa gtggtgttct tttcctcttg ggcatcatct tgctggttct gattggagtg
caaggacaaa ccccagtagt gagaaagggt cgctgttcct gcatcagcac caaccaaggg
actatccacc tacaatcctt gaaagacctt aaacaatttg ccccaagccc ttcctgcgag
aaaattgaaa tcattgctac actgaagaat ggagttcaaa catgtctaaa cccagattca
gcagatgtga aggaactgat taaaaagtgg gagaaacagg tcagccaaaa gaaaaagcaa
aagaatggga aaaaacatca aaaaaagaaa gttctgaaag ttcgaaaatc tcaacgttct
cgtcaaaaga agactaca.

In some embodiments, the polypeptide comprises the sequence of SEQ ID No. 3:

Mutant 3: Asn-CXCL9—Insertion Asn at Position 0 of the hCXCL9 N-Terminus

Protein sequence of the Asn-CXCL9 mutant:
MKKSGVLFLLGIILLVLIGVQGNTPVVRKGRCSCISTNQGT
IHLQSLKDLKQFAPSPSCEKIEIIATLKNGVQTCLNPDSAD
VKELIKKWEKQVSQKKKQKNGKKHQKKKVLKVRKSQRSRQKKTT

In some embodiments, and as an example, the sequence of the cDNA encoding hCXCL9 with an insertion of Asn at N-terminus is as set forth below at SEQ ID No: 22:

cDNA sequence of the Asn-CXCL9 mutant:
(SEQ ID No: 22)
atgaagaaaa gtggtgttct tttcctcttg ggcatcatct tgctggttct gattggagtg
caaggaaaca ccccagtagt gagaaagggt cgctgttcct gcatcagcac caaccaaggg
actatccacc tacaatcctt gaaagacctt aaacaatttg ccccaagccc ttcctgcgag
aaaattgaaa tcattgctac actgaagaat ggagttcaaa catgtctaaa cccagattca
gcagatgtga aggaactgat taaaaagtgg gagaaacagg tcagccaaaa gaaaaagcaa
aagaatggga aaaaacatca aaaaaagaaa gttctgaaag ttcgaaaatc tcaacgttct
cgtcaaaaga agactaca.

In some embodiments, the polypeptide comprises the sequence of SEQ ID No: 4:

Mutant 4: hPro-CXCL9—Insertion Pro at Position 0 of the hCXCL9 N-Terminus

Protein sequence of the hPro-CXCL9 mutant:
MKKSGVLFLLGIILLVLIGVQGPTPVVRKGRCSCISTNQGT
IHLQSLKDLKQFAPSPSCEKIEIIATLKNGVQTCLNPDSAD
VKELIKKWEKQVSQKKKQKNGKKHQKKKVLKVRKSQRSRQKKTT

In some embodiments, and as an example, the sequence of the cDNA encoding hCXCL19 with an insertion of Pro at N-terminus is as set forth below at SEQ ID No: 23:

cDNA sequence of the Pro-CXCL9 mutant:
(SEQ ID No: 23)
atgaagaaaa gtggtgttct tttcctcttg ggcatcatct tgctggttct gattggagtg
caaggaccca ccccagtagt gagaaagggt cgctgttcct gcatcagcac caaccaaggg
actatccacc tacaatcctt gaaagacctt aaacaatttg ccccaagccc ttcctgcgag
aaaattgaaa tcattgctac actgaagaat ggagttcaaa catgtctaaa cccagattca
gcagatgtga aggaactgat taaaaagtgg gagaaacagg tcagccaaaa gaaaaagcaa
aagaatggga aaaaacatca aaaaaagaaa gttctgaaag ttcgaaaatc tcaacgttct
cgtcaaaaga agactaca.

In some embodiments of the invention, any of the human modified CXCL9 polypeptide of the invention or the WT CXCL9 may be conjugated to Ig which may be IgG or a fragment thereof. In some embodiments of the invention, the IgG is without limitation, IgG-Fc: hinge-ch2-ch3.

For example, in various embodiments, the peptides are linked to the Fc portion of an immunoglobulin (e.g., to promote antibody-dependent cellular cytotoxicity (ADCC) and/or complement-dependent cytotoxicity (CDC)). In some embodiments, the CXCL9 is linked to the Fc region of an IgG antibody. In some embodiments, the CXCL9 is linked to the Fc region of a human IgG1, IgG2, IgG3 and IgG4 isotype.

As used herein, “immunoglobulin Fc region” refers to a protein that contains the heavy-chain constant region 2 (CH2) and the heavy-chain constant region 3 (CH3) of an immunoglobulin, excluding the variable regions of the heavy and light chains, the heavy-chain constant region 1 (CH1) and the light-chain constant region 1 (CL1) of the immunoglobulin. It may further include a hinge region at the heavy-chain constant region. Also, the immunoglobulin Fc region of the present invention may contain a part or all of the Fc region including the heavy-chain constant region 1 (CH1) and/or the light-chain constant region 1 (CL1), except for the variable regions of the heavy and light chains of the immunoglobulin, as long as it has an effect substantially similar to or better than that of the native form. Also, it may be a region having a deletion in a relatively long portion of the amino acid sequence of CH2 and/or CH3. That is, the immunoglobulin Fc region of the present invention may include 1) a CH1 domain, a CH2 domain, a CH3 domain and a CH4 domain, 2) a CH1 domain and a CH2 domain, 3) a CH1 domain and a CH3 domain, 4) a CH2 domain and a CH3 domain, 5) a combination of one or more domains and an immunoglobulin hinge region (or a portion of the hinge region), and 6) a dimer of each domain of the heavy-chain constant regions and the light-chain constant region.

The immunoglobulin Fc region is safe for use as a drug carrier because it is a biodegradable polypeptide that is metabolized in vivo. Also, the immunoglobulin Fc region has a relatively low molecular weight, as compared to the whole immunoglobulin molecules, and thus, it is advantageous in terms of preparation, purification and yield of the conjugate. The immunoglobulin Fc region does not contain a Fab fragment, which is highly non-homogenous due to different amino acid sequences according to the antibody subclasses, and thus it can be expected that the immunoglobulin Fc region may greatly increase the homogeneity of substances and be less antigenic in blood.

The immunoglobulin Fc region may be derived from humans or other animals including cows, goats, swine, mice, rabbits, hamsters, rats and guinea pigs, and preferably, humans. In addition, the immunoglobulin Fc region may be an Fc region that is derived from IgG, IgA, IgD, IgE, and IgM, or made by combinations thereof or hybrids thereof. Preferably, it is derived from IgG or IgM, which are among the most abundant proteins in human blood, and most preferably, from IgG which is known to enhance the half-lives of ligand-binding proteins.

IgG is divided into IgG1, IgG2, IgG3 and IgG4 subclasses, and the present invention includes combinations and hybrids thereof. Preferred are IgG1 and IgG4 subclasses, and most preferred is the Fc region of IgG4 rarely having effector functions such as CDC (complement dependent cytotoxicity).

Meanwhile, the immunoglobulin Fc region may be in the form of having native sugar chains, increased sugar chains compared to a native form or decreased sugar chains compared to the native form, or may be in a deglycosylated form. The increase, decrease or removal of the immunoglobulin Fc sugar chains may be achieved by methods common in the art, such as a chemical method, an enzymatic method and a genetic engineering method using a microorganism. Here, the removal of sugar chains from an Fc region results in a sharp decrease in binding affinity to the complement (c1q) and a decrease or loss in antibody-dependent cell-mediated cytotoxicity or complement-dependent cytotoxicity, thereby not inducing unnecessary immune responses in vivo. In this regard, an immunoglobulin Fc region in a deglycosylated or aglycosylated form may be more suitable to the object of the present invention as a drug carrier.

As used herein, “deglycosylation” means to enzymatically remove sugar moieties from an Fc region, and “aglycosylation” means that an Fc region is produced in an unglycosylated form by a prokaryote, preferably, E. coli.

Further, the immunoglobulin Fc region of the present invention includes a sequence derivative (mutant) thereof as well as a native amino acid sequence. An amino acid sequence derivative has a sequence that is different from the native amino acid sequence due to deletion, insertion, non-conservative or conservative substitution of one or more amino acid residues, or combinations thereof. For example, in IgG Fc, amino acid residues known to be important in binding, at positions 214 to 238, 297 to 299, 318 to 322, or 327 to 331, may be used as a suitable target for modification. In addition, other various derivatives are possible, including derivatives having a deletion of a region capable of forming a disulfide bond, a deletion of several amino acid residues at the N-terminus of a native Fc form, or an addition of a methionine residue to the N-terminus of a native Fc form. Furthermore, to remove effector functions, a deletion may occur in a complement-binding site, such as a C1q-binding site and an ADCC (antibody dependent cell mediated cytotoxicity) site. Techniques of preparing such sequence derivatives of the immunoglobulin Fc region are disclosed in WO 97/34631 and WO 96/32478.

The Fc region, if desired, may be modified by phosphorylation, sulfation, acrylation, glycosylation, methylation, farnesylation, acetylation, amidation or the like. In some embodiments of the invention, the CXCL9 of the invention and IgG and/or any other protein that may be used for extending the half-life of the variant of the invention in the serum are linked by a linker. In Some embodiments of the invention, the linker is a sequence of between 2-30 amino acids. In Some embodiments of the invention, the linker is a sequence of between 2-20 amino acids. In Some embodiments of the invention, the linker is a sequence of between 2-10 amino acids. In some embodiments, the linker is a poly G linker or poly GS as described herein.

An example of a heterologous amino acid sequence which may be used in accordance with this aspect of the present invention is an immunoglobulin amino acid sequence, such as the hinge and Fc regions of an immunoglobulin heavy domain (see U.S. Pat. No. 6,777,196). The immunoglobulin moiety in the chimeras of this aspect of the present invention may be obtained from IgG1, IgG2, IgG3 or IgG4 subtypes, IgA, IgE, IgD or IgM, as further discussed hereinbelow.

Typically, in such fusions the chimeric molecule will retain at least functionally active hinge and CH2 and CH3 domains of the constant region of an immunoglobulin heavy chain. Fusions can also be generated to the C-terminus of the Fc portion of a constant domain, or immediately N-terminus to the CH1 of the heavy chain or the corresponding region of the light chain.

Though it may be possible to conjugate the entire heavy chain constant region to the CXCL9 amino acid sequence of the present invention, it is preferable to fuse shorter sequences. For example, a sequence beginning at the hinge region upstream of the papain cleavage site, which defines IgG Fc chemically; residue 216, taking the first residue of heavy chain constant region to be 114, or analogous sites of other immunoglobulins, may be used in the fusion. In a particular embodiment, the CXCL9 amino acid sequence is fused to the hinge region and CH2 and CH3, or to the CH1, hinge, CH2 and CH3 domains of an IgG2, or IgG3 heavy chain (see U.S. Pat. No. 6,777,196).

For example, a nucleic acid sequence encoding a CXCL9 peptide of the present invention is ligated in frame to an immunoglobulin cDNA sequence. It will be appreciated that, ligation of genomic immunoglobulin fragments can also be used. In this case, fusion requires the presence of immunoglobulin regulatory sequences for expression. cDNAs encoding IgG heavy-chain constant regions can be isolated based on published sequence from cDNA libraries derived from spleen or peripheral blood lymphocytes, by hybridization or by polymerase chain reaction (PCR) techniques.

In some embodiments, the invention further envisages inclusion of the modified CXCL9 or the WT CXCL9 in a complex where it is attached to proteinaceous (e.g., heterologous amino acid sequence) or each of which being capable of prolonging the half-life of the composition while in circulation. Such a molecule is highly stable (resistant to in-vivo proteaolytic activity, and may be produced using common solid phase synthesis. Further recombinant techniques may still be used, whereby the recombinant peptide product is subjected to in-vitro modification (e.g., PEGylation as further described herein below).

The phrase “non-proteinaceous moiety” as used herein refers to a molecule that is attached to the above-described CXCL9 amino acid sequences. According to some embodiments the non-proteinaceous moiety may be a polymer or a co-polymer (synthetic or natural). Non-limiting examples of the non-proteinaceous moiety of the present invention include polyethylene glycol (PEG) or derivative thereof, polyvinyl pyrrolidone (PVP), albumin, divinyl ether and maleic anhydride copolymer (DIVEMA); polysialic acid (PSA) and/or poly(styrene comaleic anhydride) (SMA). Additionally, complexes which can protect CXCL9 or modified CXCL9 from the environment and thus keep its stability may be used, including, for example, liposomes or micelles are also included in the invention.

According to some embodiments of the invention, modified CXCL9 or the WT CXCL9 of the invention is attached to a non-proteinaceous moiety, which may act as a sustained-release enhancing agent. Exemplary sustained-release enhancing agents include, but are not limited to hyaluronic acid (HA), alginic acid (AA), polyhydroxyethyl methacrylate (Poly-HEMA), glyme and polyisopropylacrylamide.

Attaching the modified CXCL9 or the WT CXCL9 to other non-amino acid agents may be by covalent linking or by non-covalent complexion, for example, by complexion to a hydrophobic polymer, which can be degraded or cleaved producing a compound capable of sustained release; The association may be by the entrapment of the amino acid sequence within the other component (liposome, micelle) or the impregnation of the amino acid sequence within a polymer to produce the final peptide of the invention.

In some embodiments, the PEG derivative is N-hydroxysuccinimide (NHS) esters of PEG carboxylic acids, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. PEG-maleimide, PEG-vinylsulfone (VS), PEG-acrylate (AC) or PEG-orthopyridyl disulfide may be also used.

In some embodiments of the invention, there is provided a pharmaceutical composition comprising a modified CXCL9 polypeptide as described herein, optionally conjugated to an Ig, with or without a linker which may be a poly G or a sequence of one, two, three or more repeated units of GGGGS (SEQ ID No: 7), and a pharmaceutically acceptable carrier. In some embodiments, the modified CXCL9 polypeptide or the WT CXCL9 are linked to the non-proteinaceous moiety or the proteinaceous moiety as described above.

In some embodiments of the invention, there is provided a method of treating cancer comprising the step of administering to a subject in need a pharmaceutical composition comprising a modified CXCL9 polypeptide as described herein, optionally conjugated to an Ig, with or without a linker, and a pharmaceutically acceptable carrier.

In some embodiments of the invention, the mutated human CXCL9 that are optionally conjugated to Ig, may be administered to a subject in need in combination with another anticancer treatment, such as without being limited, such as, cellular or non-cellular immunotherapy like immune checkpoint inhibitors, cancer vaccines, conjugated antibodies, bi-specific T cell engagers, bi-specific NK cell engagers, oncolytic viruses, ‘eat me’ signals, ‘find me’ signals or others, or non-immunotherapy anti-cancer treatments, including chemotherapy, biological therapies like, for example, tyrosine kinase inhibitors, anti-angiogenic therapy, hormonal therapy, radiotherapy or surgery.

In some embodiments of the invention, there is provided a nucleic acid molecule encoding the modified CXCL9 polypeptide of the invention.

In some embodiments of the invention, there is provided a vector comprising the nucleic acid molecule encoding the modified CXCL9 polypeptide of the invention. The vector being an expression vector, further comprises one or more regulatory sequences.

In some embodiments of the invention, the nucleic acid molecule of the invention or the vector may be used for use in treating cancer in a subject in need thereof.

In some embodiments of the invention, there is provided a host cell comprising the nucleic acid molecule of the invention. In some embodiments of the invention, there is provided host cells transformed or transfected with the vector of the invention. In some embodiments of the invention, there is provided a host cell comprising the modified CXCL9 polypeptide of the invention.

In some embodiments of the invention, there is provided a method of producing the modified CXCL9 polypeptide, the method comprising: (i) culturing the host cells comprising the nucleic acids encoding the modified CXCL9 polypeptide under conditions such that the polypeptide comprising the modified CXCL9 is expressed; and (ii) optionally recovering the modified CXCL9 from the host cells or from the culture medium.

According to some embodiments, any suitable route of administration to a subject may be used for the nucleic acid, polypeptide or the composition of the present invention, including but not limited to, local and systemic routes. Exemplary suitable routes of administration include, but are not limited to: orally, intra-nasally, parenterally, intravenously, topically, enema or by inhalation. According to another embodiment, systemic administration of the composition is via an injection. For administration via injection, the composition may be formulated in an aqueous solution, for example in a physiologically compatible buffer including, but not limited, to Hank's solution, Ringer's solution, or physiological salt buffer. Formulations for injection may be presented in unit dosage forms, for example, in ampoules, or in multi-dose containers with, optionally, an added preservative.

According to another embodiment, administration systemically is through a parenteral route. According to some embodiments, parenteral administration is administration intravenously, intra-arterially, intramuscularly, intraperitoneally, intradermally, intravitreally, or subcutaneously. Each of the abovementioned administration routes represents a separate embodiment of the present invention. According to another embodiment, parenteral administration is performed by bolus injection. According to another embodiment, parenteral administration is performed by continuous infusion. According to some embodiments, preparations of the composition of the invention for parenteral administration include sterile aqueous or non-aqueous solutions, suspensions, or emulsions, each representing a separate embodiment of the present invention. Non-limiting examples of non-aqueous solvents or vehicles are propylene glycol, polyethylene glycol, vegetable oils such as olive oil and corn oil, gelatin, and injectable organic esters such as ethyl oleate.

According to another embodiment, parenteral administration is transmucosal administration. According to another embodiment, transmucosal administration is transnasal administration. For transmucosal administration, penetrants appropriate to the barrier to be permeated are used in the formulation. Such penetrants are generally known in the art. The preferred mode of administration will depend upon the particular indication being treated and will be apparent to one of skill in the art.

Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

According to another embodiment, compositions formulated for injection may be in the form of solutions, suspensions, dispersions or emulsions in oily or aqueous vehicles, and may contain formulatory agents such as suspending, stabilizing, and/or dispersing agents. Non-limiting examples of suitable lipophilic solvents or vehicles include fatty oils such as sesame oil, or synthetic fatty acid esters such as ethyl oleate or triglycerides.

According to another embodiment, the composition is administered intravenously, and is thus formulated in a form suitable for intravenous administration. According to another embodiment, the composition is administered intra-arterially, and is thus formulated in a form suitable for intra-arterial administration. According to another embodiment, the composition is administered intramuscularly, and is thus formulated in a form suitable for intramuscular administration.

According to another embodiment, administration systemically is through an enteral route. According to another embodiment, administration through an enteral route is buccal administration. According to another embodiment, administration through an enteral route is oral administration. According to some embodiments, the composition is formulated for oral administration.

According to some embodiments, oral administration is in the form of hard or soft gelatin capsules, pills, capsules, tablets, including coated tablets, dragees, elixirs, suspensions, liquids, gels, slurries, syrups or inhalations and controlled release forms thereof.

According to some embodiments, suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

In some embodiments, if desired, disintegrating agents, such as cross-linked polyvinyl pyrrolidone, agar, or alginic acid or a salt thereof, such as sodium alginate, may be added. Capsules and cartridges of, for example, gelatin, for use in a dispenser may be formulated containing a powder mix of the composition of the invention and a suitable powder base, such as lactose or starch.

According to some embodiments, solid dosage forms for oral administration include capsules, tablets, pill, powders, and granules. In such solid dosage forms, the composition of the invention is admixed with at least one inert pharmaceutically acceptable carrier such as sucrose, lactose, or starch. Such dosage forms can also comprise, as it normal practice, additional substances other than inert diluents, e.g., lubricating, agents such as magnesium stearate. In the case of capsules, tablets and pills, the dosage forms may also comprise buffering, agents. Tablets and pills can additionally be prepared with enteric coatings.

In some embodiments, liquid dosage forms for oral administration may further contain adjuvants, such as wetting agents, emulsifying and suspending agents, and sweetening, flavoring and perfuming agents. According to some embodiments, enteral coating of the composition is further used for oral or buccal administration. The term “enteral coating”, as used herein, refers to a coating which controls the location of composition absorption within the digestive system. Non-limiting examples for materials used for enteral coating are fatty acids, waxes, plant fibers or plastics.

According to some embodiments, administering is administering topically. According to some embodiments, the composition is formulated for topical administration. The term “topical administration”, as used herein, refers to administration to body surfaces. Non-limiting examples of formulations for topical use include cream, ointment, lotion, gel, foam, suspension, aqueous or cosolvent solutions, salve and sprayable liquid form. Other suitable topical product forms for the compositions of the present invention include, for example, emulsion, mousse, lotion, solution and serum.

According to some embodiments, the administration may include any suitable administration regime, depending, inter alia, on the medical condition, patient characteristics, administration route, and the like. In some embodiments, administration may include administration twice daily, every day, every other day, every third day, every fourth day, every fifth day, once a week, once every second week, once every third week, once every month, and the like.

According to some embodiments, the modified CXCL9 polypeptide, the nucleic acid encoding the same, and/or the composition comprising the polypeptide or the nucleic acid molecules, when used for used for treating cancer may be used in combination with other therapeutic agents. The components of such combinations may be administered sequentially or simultaneously/concomitantly in separate or combined pharmaceutical formulations by any suitable administration route.

According to some embodiments, there are provided kits comprising the modified CXCL9 polypeptide and/or the nucleic acid molecule encoding the same and/or the composition as disclosed herein. Such a kit can be used, for example, in the treatment of cancer.

In some embodiments, the pharmaceutical compositions of the invention may be administrated in combination with other immune checkpoint blockers, such as without being limited, anti PD-1. In some embodiments, the combined treatment may be used for tumors in which anti PD-1 is not successfully significant by itself, such as glioma and triple negative breast cancer

In the description and claims of the application, the words “include” and “have”, and forms thereof, are not limited to members in a list with which the words may be associated.

As used herein, the term comprising includes the term consisting of.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Experimental Methods

Construction of pSecTag-Ig Vector:

cDNA encoding the constant region of Fc (Hinge-CH2-CH3) of mouse IgG1 was constructed from RNA extracted from mouse splenocytes that were cultured for 96 h in the presence of lipopolysaccharide (LPS) and mouse-Interleukin 4 (mIL-4). The primers used for this reaction were 5′CTCGAGGTGCCCAGGGATTGTGGTTG-3′ (sense) (SEQ ID No. 13) and 5′-GGGCCCTTTACCA GGAGA GTGGGAGA-3′(anti-sense) (SEQ ID No. 14). The PCR product was then digested with XhoI and ApaI, and ligated into the mammalian expression/secretion vector pSecTag2/Hygro B (Invitrogen). Next, the new construct underwent cleavage with Nhe1 and Xho1 to remove the original mouse NF-kappa leader sequence found in the original pSecTag2/Hygro B vector. These two steps revealed a modified pSecTag2/Hygro B vector named pSecTag-Ig lacking a signal peptide and include the Hinge-CH2-CH3 of the mouse IgG1 located immediately before the sequences coding for the c-myc and 5 residues of histidine built in the original pSectag-hygro b vector.

Cloning of the Chemokines into the pSecTag-Ig Vector:

The sequences of chemokines (naïve or mutated sequences) were provided by Rhenium. The chemokines sequences composed of the original signal peptide, the coding region of the chemokine and the cleavage site sequences of the restriction enzyme Nhe1 (GCTAGC) (SEQ ID No: 15) and Xho1 (CTCGAG) (SEQ ID No. 16) at the 5′ and 3′, correspondently. The chemokines were subcloned into the vector containing the mouse IgG1 fragment after digestion with Nhe1 and xho1. The fused fragments were sequenced by dideoxynucleotide sequencing in our facility (Sequenase version 2; Millipore).

### Expression of the Constructs in 293T and CH0-DG-44

The constructs were transfected into HEK-293T for transient expression. Next were transfected into Chinese hamster ovary dhfr−/− (DG44) cells (provided by L. Chasin, Columbia University, New York, N.Y.). Stable cell lines producing the chemokines were generated in the DG-44 cells. The production of the chemokines improved by selection with gradually increasing concentrations of methotrexate. The fusion protein was purified from the culture medium by a Nickle-column Ni-NTA (Thermo-scientific).

The following sequences were used in the experiments with mouse-CXCL9 protein sequence and mCXCL9-polyG protein sequence.

mouse-CXCL9 protein sequence
(SEQ ID NO: 17)
MKSAVLFLLGIIFLEQCGVRGTLVIRNARCSCISTSRGTIHYKS
LKDLKQFAPSPNCNKTEIIATLKNGDQTCLDPDSANVKKLMKEW
EKKISQKKKQKRGKKHQKNMKNRKPKTPQSRRRSRKTT
mCXCL9-polyGS protein sequence
(SEQ ID NO: 18)
MKSAVLFLLGIIFLEQCGVRGTLVIRNARCSCISTSRGTIHYKS
LKDLKQFAPSPNCNKTEIIATLKNGDQTCLDPDSANVKKLMKEW
EKKISQKKKQKRGKKHQKNMKNRKPKTPQSRRRSRKTTGGGGSGGGGSG
GGGS

Further, the following nucleic acid sequences were used:

Mouse IgG1-Fc (Hinge region-CH2-CH3)
(SEQ ID NO: 19)
GTGCCCAGGGATTGTGGTTGTAAGCCTTGCATATGTACAGTCCCAGAAGTATCAT
CTGTCTTCATCTTCCCCCCAAAGCCCAAGGATGTGCTCACCATTACTCTGACTCCT
AAGGTCACGTGTGTTGTGGTAGACATCAGCAAGGATGATCCCGAGGTCCAGTTC
AGCTGGTTTGTAGATGATGTGGAGGTGCACACAGCTCAGACGCAACCCCGGGAG
GAGCAGTTCAACAGCACTTTCCGCTCAGTCAGTGAACTTCCCATCATGCACCAGG
ACTGGCTCAATGGCAAGGAGTTCAAATGCAGGGTCAACAGTGCAGCTTTCCCTG
CCCCCATCGAGAAAACCATCTCCAAAACCAAAGGCAGACCGAAGGCTCCACAGG
TGTACACCATTCCACCTCCCAAGGAGCAGATGGCCAAGGATAAAGTCAGTCTGA
CCTGCATGATAACAGACTTCTTCCCTGAAGACATTACTGTGGAGTGGCAGTGGAA
TGGGCAGCCAGCGGAGAACTACAAGAACACTCAGCCCATCATGGACACAGATGG
CTCTTACTTCGTCTACAGCAAGCTCAATGTGCAGAAGAGCAACTGGGAGGCAGG
AAATACTTTCACCTGCTCTGTGTTACATGAGGGCCTGCACAACCACCATACTGAG
AAGAGCCTCTCCCACTCTCCTGGTAAA
WT-mCXCL9-Ig: Mouse-CXCL9 (small letters) fused with mouse IgG1-Fc
(capital letters)
(SEQ ID NO: 20)
atgaagtccg ctgttctttt ccttttgggc atcatcttcc tggagcagtg tggagttcga
ggaaccctag tgataaggaa tgcacgatgc tcctgcatca gcaccagccg aggcacgatc
cactacaaat ccctcaaaga cctcaaacag tttgccccaa gccccaattg caacaaaact
gaaatcattg ctacactgaa gaacggagat caaacctgcc tagatccgga ctcggcaaat
gtgaagaagc tgatgaaaga atgggaaaag aagatcagcc aaaagaaaaa gcaaaagagg
gggaaaaaac atcaaaagaa catgaaaaac agaaaaccca aaacacccca aagtcgtcgt
cgttcaagga agactaca
GTGCCCAGGGATTGTGGTTGTAAGCCTTGCATATGTACAGTCCCAGAAGTATCAT
CTGTCTTCATCTTCCCCCCAAAGCCCAAGGATGTGCTCACCATTACTCTGACTCCT
AAGGTCACGTGTGTTGTGGTAGACATCAGCAAGGATGATCCCGAGGTCCAGTTC
AGCTGGTTTGTAGATGATGTGGAGGTGCACACAGCTCAGACGCAACCCCGGGAG
GAGCAGTTCAACAGCACTTTCCGCTCAGTCAGTGAACTTCCCATCATGCACCAGG
ACTGGCTCAATGGCAAGGAGTTCAAATGCAGGGTCAACAGTGCAGCTTTCCCTG
CCCCCATCGAGAAAACCATCTCCAAAACCAAAGGCAGACCGAAGGCTCCACAGG
TGTACACCATTCCACCTCCCAAGGAGCAGATGGCCAAGGATAAAGTCAGTCTGA
CCTGCATGATAACAGACTTCTTCCCTGAAGACATTACTGTGGAGTGGCAGTGGAA
TGGGCAGCCAGCGGAGAACTACAAGAACACTCAGCCCATCATGGACACAGATGG
CTCTTACTTCGTCTACAGCAAGCTCAATGTGCAGAAGAGCAACTGGGAGGCAGG
AAATACTTTCACCTGCTCTGTGTTACATGAGGGCCTGCACAACCACCATACTGAG
AAGAGCCTCTCCCACTCTCCTGGTAAA
Mouse mutant 1-mCXCL9-polyG-Ig: mouse CXCL9 with the polyGS at its
C-terminus fused to the mouse IgG1-Fc
Mouse-CXCL9 (small letters) + polyG (Italics) + IgG (capital letters)
(SEQ ID NO: 21)
atgaagtccg ctgttctttt ccttttgggc atcatcttcc tggagcagtg tggagttcga
ggaaccctag tgataaggaa tgcacgatgc tcctgcatca gcaccagccg aggcacgatc
cactacaaat ccctcaaaga cctcaaacag tttgccccaa gccccaattg caacaaaact
gaaatcattg ctacactgaa gaacggagat caaacctgcc tagatccgga ctcggcaaat
gtgaagaagc tgatgaaaga atgggaaaag aagatcagcc aaaagaaaaa gcaaaagagg
gggaaaaaac atcaaaagaa catgaaaaac agaaaaccca aaacacccca aagtcgtcgt
cgttcaagga agactaca
GGCGGAGGTGGCTCTGGCGGTGGCGGATCGGGCGGAGGTGGCTCTGTGCCCAGGG
ATTGTGGTTGTAAGCCTTGCATATGTACAGTCCCAGAAGTATCATCTGTCTTCATC
TTCCCCCCAAAGCCCAAGGATGTGCTCACCATTACTCTGACTCCTAAGGTCACGT
GTGTTGTGGTAGACATCAGCAAGGATGATCCCGAGGTCCAGTTCAGCTGGTTTGT
AGATGATGTGGAGGTGCACACAGCTCAGACGCAACCCCGGGAGGAGCAGTTCAA
CAGCACTTTCCGCTCAGTCAGTGAACTTCCCATCATGCACCAGGACTGGCTCAAT
GGCAAGGAGTTCAAATGCAGGGTCAACAGTGCAGCTTTCCCTGCCCCCATCGAG
AAAACCATCTCCAAAACCAAAGGCAGACCGAAGGCTCCACAGGTGTACACCATT
CCACCTCCCAAGGAGCAGATGGCCAAGGATAAAGTCAGTCTGACCTGCATGATA
ACAGACTTCTTCCCTGAAGACATTACTGTGGAGTGGCAGTGGAATGGGCAGCCA
GCGGAGAACTACAAGAACACTCAGCCCATCATGGACACAGATGGCTCTTACTTC
GTCTACAGCAAGCTCAATGTGCAGAAGAGCAACTGGGAGGCAGGAAATACTTTC
ACCTGCTCTGTGTTACATGAGGGCCTGCACAACCACCATACTGAGAAGAGCCTCT
CCCACTCTCCTGGTAAA

### Example 1

### CXCL9 Inhibits Proliferation of Hela (Human Cervical Cancer).

At first, an experiment was made to asses if the addition of murine CXCL9 to cultured Hela cell line affects their proliferation/viability rate (XTT assay)

Hela Cells were seeded in 96 well plate in RPMI medium supplemented with 10% FCS, penicillin, streptomycin and glutamic acid (2×10⁴ cells per well). 24 h after seeding the medium was replaced with fresh one supplemented with different concentration of mouse CXCL9 (Peprotech Cat #250-18) as indicated in the graph, or without treatment (WO). 24 hours later XTT assay was performed according to the manufactory instructions (Biological Industries, cat #20-300-1000). The OD measurements were taken after 2 hours incubation with the XTT substrate. The assay was performed with six well for each treatment.

Two methods by which CXCL9 is stabilized and be effectively used for cancer immunotherapy are suggested: the first includes addition of poly GS as a linker that includes three tandem repeats of GGGGS at the C-terminal site of CXCL9 as a linker between CXCL9 and the Fc (i.e. CXCL9-poly GS), and the other is insertion of an amino acid, such as glutamine (Gln), Asparagine (Asn), Proline (Pro), pyroglutamate or glutamic acid, or any other single or more amino acids at the N-terminus site of CXCL9 in order to prevent the ability of DPP4 to cleave the chemokine at the proline in position 2 without affecting the open reading frame of the chemokine, resulting in a chemokine variant that is stable against proteolytic cleavage of the CXCL9 with DPP4 and is functional as an anti-cancer drug.

### Results

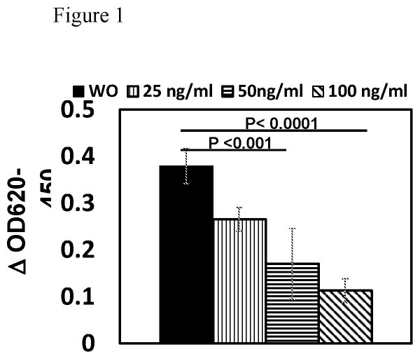

FIG. 1 shows results of an experiment in which the ability of CXCL9-Ig (murine CXLC9 linked to IgG-Fc: hinge-ch2-ch3) to inhibit melanoma growth versus its isotype matched IgG in an experimental mice melanoma model was assessed. This includes tumor growth rate (panel A), scattered analyses of a single time point (day 17) (panel B) and mortality after therapy has been terminated (panel C). As can be seen, CXCL9-Ig inhibits melanoma growth much more efficiently than its isotype matched IgG.

### Example 2

Tumor Progression and Mortality Analysis in C57Bl/6 Mice Treated with CXCL9-Ig Versus the Control Group

Mice (14 females at age of 8 weeks) were injected subcutaneously with 3.5×10⁵ Ret melanoma pre-line at the right flank. On day 3, mice were separated into 2 groups of 7 females each. Each group was treated (3 time a week, 40 μg/mouse) with either CXCL9-Ig (i.e. mCXCL9 with an IgG-Fc, or with isotype matched control IgG. Every other day, tumor size was measured using a scientific caliper, by an observer blind to the experimental protocol. On day 9 a single mouse with no tumor development has been subtracted from each group. On day 23, therapy was terminated, and mice were continued to be followed for mortality.

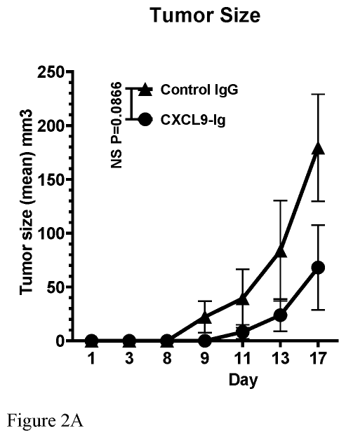

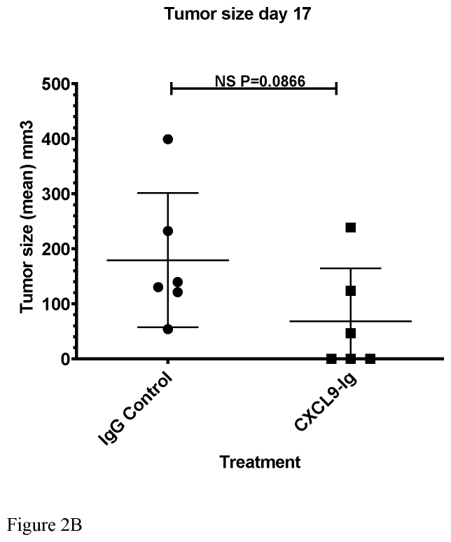

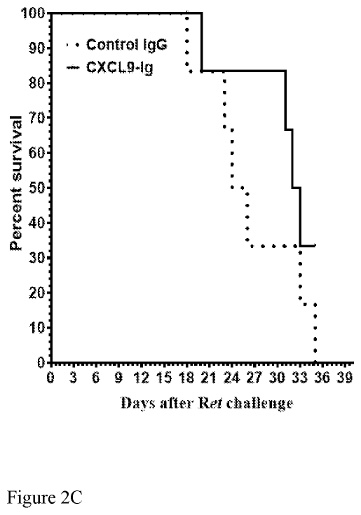

FIG. 2A shows tumor size as mean size ±SD (length×width×height)×0.52. FIG. 2B shows scattered analyses on day 17. Panel FIG. 2C shows mortality curve.

This experiment shows that the treatment with mCXCL9-Ig attenuates the primary tumor development rate and significantly increases mice survival

*P≤0.05 was considered as significant.

### Example 3

Modified CXCL9-Ig with Addition of Either Glutamine (Gin) or Asparagine (Asn) at the N-Terminus Site Override Proteolytic Cleavage by DPP4

In human, both CXCL10 (1-77 amino acids) and CXCL9 (1-103 amino acids) are subjected to exo-proteolytic post translational modification (PTM) by the exo-protease Dipeptidyl peptidase 4 (DPP4, also known as CD26).

DPP4 recognizes proline at position 2 and cleaves at its C-terminus resulting in a truncated non-functional CXCL10 (3-77 amino acids) or CXCL9 (3-103). These truncated non-functional CXCL9 or CXCL10 may also act as potent CXCR3 antagonists. At tumor sites DPP4 is largely produced and therefore is likely to play a major role in targeting CXCL9 and CXCL10.

The exo-protease cleavage site is X₁-P₂ (X-any amino acid at the N-terminus, P-proline at position 2). Therefore, an insertion of an additional single amino acid at the N-terminus of these chemokines move the proline to position 3 and protect these chemokines from proteolytic cleavage by DPP4.

### Generation of Gln-CXCL9-Ig and Asn-CXCL9-Ig

Gln-CXCL9-Ig and Asn-CXCL9-Ig are generated as follows: The sequence of the “mutated CXCL9” was cloned in the pcDNA3.1. The “mutated sequences” included the complete sequence of the CXCL9 including the signal peptide. The sequence flanked with the NheI (restriction enzyme) cleavage site at the 5′ end and the XhoI (restriction enzyme) cleavage site sequence at the 3′ end. Sequence of Gln, Asn or Pro codons were placed immediately after the glycine at position 22 (the cleavage site of the signal peptide) to assure that it is placed at position 0 of the N-terminus CXCL9. The mutated sequences were cleaved with NheI and XhoI to remove it from the pcDNA 3.1 plasmid and were recloned in pSecTag-Ig vector (describes previously) to form the conjugation of the “mutated CXCL9” with the mouse IgG1-Fc.

### Examination of Ca++ Flux:

In order to assessed whether the mutants still able to bind the receptor (CXCR3) and activate it, Ca++ flux is measured:

human and mouse CXCL9 (purchased from Peprotech, USA), human CXCL9-Ig, Gln-CXCL9-Ig and Asn-CXCL9 are added to CHO-K1 cells that overexpress both human CXCR3A and Apoaequorin (oxidation of Apoaequorin release aequorin, a calcium-sensitive bioluminescent protein) In these cells upon coupling of the receptor with its ligands (GPCR), calcium channels are activated and stimulate calcium influx. Elevated Ca++ in the cells activates Aequorin that emits blue light when bound to calcium ions and serve as indicator for occurrence of calcium influx. The ability of CXCL9-Ig, Gln-CXCL9-Ig and Asn-CXCL9-Ig (all the Ig used in the examples are IgG-Fc: hinge-ch2-ch3) to induce Ca++ flux is measured in this system. The protocol includes addition of 0.1 or 0.2 ug/ml of each detected chemokine. Ca++ flux is be determined 0, 5, 10, 15, 20, 25, 30 and 35 seconds after each of the modified chemokine is added. Luminescence reader records levels of Ca++ flux as Luminescence units.

### Example 4

### Examining the Ability of Mice DPP4 to Cleave the Human CXCL9-Ig and its Mutants

Next it is examined whether the mouse recombinant DPP4 cleaves human CXCL9, hCXCL9-Ig and the mutated hCXCL9-Ig. The addition of the mouse DPP4 to recombinant human CXCL9 is tested in terms of whether it restricts it to a non-active compound. If the response is positive, wild type C57Bl/6 mice are used in the in vivo experiments. If not, transgenic mice overexpressing human DPP4 (Caygen https://www.cyagen.com/us/en/service/transgenic-mice.html) are used. Either way, mice are engrafted with ret melanoma cell line.

### Example 5

### In Vivo Validation of the Efficiency of CXCL9-Ig (Gln) and CXCL9-Ig (Asn) in a Mouse Model of Melanoma

The basic experimental set-up and administration protocol is according to Example 2. It is an immunocompetent model of melanoma in C57Bl/6 mice in which Ret pre-line is engrafted subcutaneously (350,000 cells per mouse). On day 3, only mice with positive tumors are re-grouped and subjected to repeated administrations (3 times a week, 40 μg/mouse) of CXCL9-Ig, CXCL9-Ig (Gln), CXCL9-Ig (Asn) or control IgG and monitored for tumor growth, and later for mortality, by an observer blind to the experimental protocol. CXCL9-Ig (Gln), CXCL9-Ig (Asn) is tested in comparison to the WT CXCL9-Ig in restraining cancer development.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1. A modified CXCL9 polypeptide, comprising an insertion of an additional amino acid at the N-terminus of a corresponding wild type CXCL9.

2. (canceled)

3. The modified CXCL9 polypeptide of claim 1, wherein the additional amino acid is glutamine, pyroglutamate or glutamic acid, asparagine or proline.

4. (canceled)

5. The modified CXCL9 polypeptide of claim 1 having an amino acid sequence as denoted by any one of SEQ ID NOs: 1, 2, 3 and 4.

6. The modified CXCL9 polypeptide of claim 1, wherein the modified CXCL9 polypeptide is linked to an immunoglobulin (Ig) molecule or a fragment of an Ig molecule.

7. The modified CXCL9 of claim 6, wherein the immunoglobulin is IgG-Fc: hinge-ch2-ch3 denoted by SEQ ID. No. 5.

8. The modified CXCL9 of claim 1, further comprising a linker between the modified CXCL9 and the immunoglobulin molecule or the fragment thereof.

9. The modified CXCL9 polypeptide of claim 1, wherein the immunoglobulin or the fragment thereof is of human origin.

10. (canceled)

11. (canceled)

12. The modified CXCL9 polypeptide of claim 1 capable of binding to CXCR3 receptor and/or inducing CD8+ T cells.

13. (canceled)

14. A fusion protein comprising CXCL9 polypeptide conjugated to an immunoglobulin molecule or a fragment of an Ig molecule.

15. The fusion protein of claim 14, wherein the immunoglobulin or the fragment thereof is IgG-Fc: hinge-ch2-ch3.

16. The fusion protein of claim 14, wherein the CXCL9, the immunoglobulin molecule or a fragment thereof are of human origin.

17. The fusion protein of claim 14, further comprising a linker between the CXCL9 and the immunoglobulin or the fragment thereof.

18. (canceled)

19. (canceled)

20. The fusion protein of claim 14 capable of binding to CXCR3 receptor and/or inducing CD8+ T cells.

21. (canceled)

22. (canceled)

23. A pharmaceutical composition comprising the modified CXCL9 polypeptide of claim 1 and a pharmaceutically acceptable carrier.

24. (canceled)

25. A method of treating cancer in a subject in need thereof, the method comprising administering to the subject in need thereof a therapeutically amount of the pharmaceutical composition of claim 23.

26. A nucleic acid molecule encoding the modified CXCL9 polypeptide of claim 1.

27. A vector comprising the nucleic acid molecule of claim 26.

28. (canceled)

29. (canceled)

30. A method of treating cancer in a subject in need thereof, the method comprising administering to the subject in need thereof a therapeutically amount of the nucleic acid molecule according to claim 26.

31.-34. (canceled)


SEQUENCE LISTING (24 sequences)

SEQ ID NO: 1 - protein, 126 aa, Artificial Sequence
  Synthetic Sequence
  misc_feature (23)..(23): Xaa can be any naturally occurring amino acid
MKKSGVLFLLGIILLVLIGVQGXTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 2 - protein, 126 aa, Artificial Sequence
  Synthetic Sequence
MKKSGVLFLLGIILLVLIGVQGQTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 3 - protein, 126 aa, Artificial Sequence
  Synthetic Sequence
MKKSGVLFLLGIILLVLIGVQGNTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 4 - protein, 126 aa, Artificial Sequence
  Synthetic Sequence
MKKSGVLFLLGIILLVLIGVQGPTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 5 - protein, 232 aa, Artificial Sequence
  Synthetic Sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 15 aa, Artificial Sequence
  Synthetic Sequence
GGGGSGGGGSGGGGS

SEQ ID NO: 7 - protein, 5 aa, Artificial Sequence
  Synthetic Sequence
GGGGS

SEQ ID NO: 8 - protein, 125 aa, Artificial Sequence
  Synthetic Sequence
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 9 - DNA, 375 bp, Artificial Sequence
  Synthetic Sequence
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 10 - protein, 140 aa, Artificial Sequence
  Synthetic Sequence
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTTGGGGSGGGGSGGGGS

SEQ ID NO: 11 - DNA, 420 bp, Artificial Sequence
  Synthetic Sequence
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacaggcggaggtggctctggcggtggcggatcgggcggaggtggctct

SEQ ID NO: 12 - DNA, 378 bp, Artificial Sequence
  Synthetic Sequence
atgaagaaaagtggtgttcttttcctcttgggcatcatcttgctggttctgattggagtgcaaggacaaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 13 - DNA, 26 bp, Artificial Sequence
  Synthetic Sequence
ctcgaggtgcccagggattgtggttg

SEQ ID NO: 14 - DNA, 26 bp, Artificial Sequence
  Synthetic Sequence
gggccctttaccaggagagtgggaga

SEQ ID NO: 15 - DNA, 6 bp, Artificial Sequence
  Synthetic Sequence
gctagc

SEQ ID NO: 16 - DNA, 6 bp, Artificial Sequence
  Synthetic Sequence
ctcgag

SEQ ID NO: 17 - protein, 126 aa, Artificial Sequence
  Synthetic Sequence
MKSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTT

SEQ ID NO: 18 - protein, 141 aa, Artificial Sequence
  Synthetic Sequence
MKSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTTGGGGSGGGGSGGGGS

SEQ ID NO: 19 - DNA, 681 bp, Artificial Sequence
  Synthetic Sequence
gtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 20 - DNA, 1059 bp, Artificial Sequence
  Synthetic Sequence
atgaagtccgctgttcttttccttttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacagtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 21 - DNA, 1104 bp, Artificial Sequence
  Synthetic Sequence
atgaagtccgctgttcttttccttttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacaggcggaggtggctctggcggtggcggatcgggcggaggtggctctgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 22 - DNA, 378 bp, Artificial Sequence
  Synthetic Sequence
atgaagaaaagtggtgttcttttcctcttgggcatcatcttgctggttctgattggagtgcaaggaaacaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 23 - DNA, 378 bp, Artificial Sequence
  Synthetic Sequence
atgaagaaaagtggtgttcttttcctcttgggcatcatcttgctggttctgattggagtgcaaggacccaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 24 - DNA, 696 bp, Artificial Sequence
  Synthetic Sequence
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa